(19) United States
(12) Patent Application Publication
(10) Pub. No.: US 2026/0325691 A1
(43) Pub. Date: Oct. 22, 2026

(54) Ultraviolet room disinfecting device with adjustable shielding

(71) Applicant: LeBlanc Investments LLC, Rochester, MN (US)

(72) Inventor: Danielle LeBlanc, Rochester, MN (US)

(73) Assignee: LeBlanc Investments LLC, Rochester, MN (US)

(21) Appl. No.: 19/722,452

(22) Filed: Jun. 26, 2026

Related U.S. Application Data
(63) Continuation-in-part of application No. 19/341,278, filed on Sep. 26, 2025.
(60) Provisional application No. 63/700,635, filed on Sep. 28, 2024.

(51) Int. Cl. A61L 2/10; A61L 2/24; A61L 2/26; A61L 103/75

(52) U.S. Cl. CPC A61L 2/10; A61L 2/24; A61L 2/26; A61L 2103/75; A61L 2202/11; A61L 2202/121; A61L 2202/16

(57) ABSTRACT

An ultraviolet (UV) room-sterilization device includes an upper portion with germicidal lamps and movable shield walls mounted by multi-hinged arms. The walls move from a closed position to selected open positions to form a shadow area that protects occupants or equipment while permitting disinfection elsewhere. Hinge-position sensors and/or UV detectors generate a live emission map used to classify motion detected by onboard sensors: movement within the shadow is permitted, movement toward its boundary triggers a warning, and movement in exposed regions causes immediate lamp shutoff. A timer provides delayed start and cycle limits with code-based authorization. Embodiments include circumferential or sector lamps, retractable walls slotted into the housing, and floor, tabletop, or handheld form factors, enabling safe, targeted disinfection in occupied spaces.

### TECHNICAL FIELD

The present application relates to the field of ultraviolet room disinfecting.

### BRIEF DESCRIPTION OF THE DRAWINGS

FIG. 1 is a front plan view of a first device.

FIG. 2 is a top plan view of the device of FIG. 1.

FIG. 3 is a front plan view of the device of FIG. 1 with the left and right shield walls open.

FIG. 4 is a top plan view of the device of FIG. 1 with the left and right shield walls in the configuration of FIG. 3.

FIG. 5 is a top plan view of the device of FIG. 1 with the left and right shield walls in a second configuration.

FIG. 6 is a top plan view of the device of FIG. 1 with the left and right shield walls in the configuration of FIG. 5, showing ultraviolet light rays emanating from the device.

FIG. 7 is a top plan view of a second device, in which bulbs are arranged around the circumference of the upper portion.

FIG. 8 is a top plan view of the second device showing ultraviolet light rays emanating from the device.

FIG. 9 is a top plan view of the second device with the left and right shield walls in an alternative configuration, showing ultraviolet light rays emanating from the device.

FIG. 10 is a top plan view of a third device.

FIG. 11 is a front plan view of a fourth device.

FIG. 12 is a front plan view of a fifth, tabletop device.

FIG. 13 is a front plan view of a sixth, handheld device.

FIG. 14 is a front plan view of a seventh device with the left and right shield walls in an open configuration.

FIG. 15 is a top plan view of the seventh device of FIG. 14.

FIG. 16 is a top plan view of the seventh device of FIG. 14 with the left and right shield walls in a closed configuration.

FIG. 17 is a top plan view of the seventh device of FIG. 14 with the left and right shield walls in an operating configuration, showing ultraviolet light rays emanating from the device.

FIG. 18 is a schematic view of the controller that operates a device of the type described herein.

FIG. 19 is a schematic view of a UV sterilization device positioned in a room with multiple remote UV sensors associated with a patient.

FIG. 20 is a flow chart showing a method for using a UV sterilization device.

### DETAILED DESCRIPTION

### Overall Apparatus

Ultraviolet (UV) sterilization is a well-established technique for killing or inactivating microorganisms such as bacteria, viruses, and fungi. UV light in a germicidal range can damage the DNA or RNA of microorganisms, prevent replication and rendering them harmless. In many conventional implementations, germicidal UV light is provided at or around a wavelength of 254 nanometers, although other UV wavelengths may also be used for sterilization or disinfection depending on the light source, target microorganism, exposure time, and intended application. The effectiveness of UV sterilization depends on the dosage, which is a function of light intensity and exposure time. In hospital, clinic, and other medical settings, UV sterilization devices are often deployed to disinfect rooms, equipment, and surfaces between patient uses. Fixed or portable UV devices can bathe the air and exposed surfaces in germicidal light, achieving rapid sterilization without the need for chemical agents.

While UV light can be highly effective for sterilization, it also poses risks to humans. Direct exposure to germicidal wavelengths can cause skin burns and eye damage, including photokeratitis. Because of these hazards, many existing UV sterilization devices are used only when a room is unoccupied. This limitation can be problematic in environments where rapid or continuous disinfection is desirable, such as in areas with high patient turnover or in the vicinity of infectious individuals. A device that delivers effective UV sterilization while protecting occupants from harmful exposure can provide significant advantages in clinical and other settings.

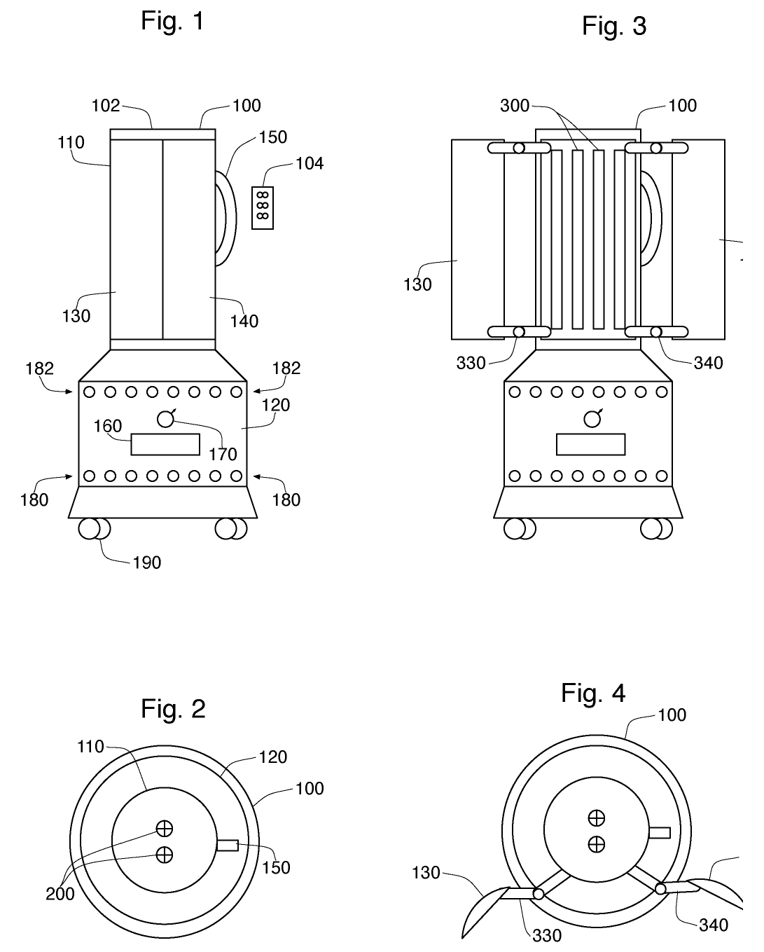

FIGS. 1 and 2 illustrate such a device 100, which delivers germicidal UV light while incorporating safety features to protect people in the vicinity. The device 100 has a housing 102 that includes an upper portion 110 supported on a base portion 120. Both the upper portion 110 and the base portion 120 have a circular cross-section, as shown in FIG. 2, with the upper portion 110 having a smaller diameter than the base portion 120. The upper portion 110 houses an ultraviolet light source (not shown in these figures) that emits germicidal light during operation. In the configuration shown, a left shield wall 130 and a right shield wall 140 are positioned in a closed position against the upper portion 110. In this embodiment, even if the UV light source were turned on, the closed shield walls 130, 140 would block all or substantially all UV light from escaping.

The device 100 may be fabricated with a stainless-steel chassis and exterior housing to provide structural rigidity, impact resistance, and resistance to hospital disinfectants, while permitting smooth, cleanable surfaces suitable for clinical environments; other metals, such as aluminum, or coated polymers can be substituted without departing from the design. The shield walls 130 and 140 are preferably formed of UV-attenuating plastic, such as an orange/amber polycarbonate or acrylic of the type used as a barrier with dental UV curing lights, sized and configured to block direct germicidal rays while remaining sufficiently translucent in the visible range for operator situational awareness; alternative UV-barrier materials may be used. The shield panels may be framed with stainless-steel or aluminum edge members for hinge attachment, include abrasion-resistant or anti-yellowing coatings, and be removable or replaceable for service. These material choices support durability, cleanability, and safety while enabling the adjustable shielding described herein. In some embodiment, the shield walls 130 and 140 may use an UV-blocking coating, such as those available from Optical Coating Laboratory Inc. in Santa Rosa, CA. Such coatings may be applied on a polycarbonate wall or a wall made of different materials. In some embodiments, the shield material or coating may produce a visible glow when exposed to ultraviolet radiation. The visible glow may result from fluorescence of the shield material or coating, scattering at the shield surface or edges, or other optical interaction between the ultraviolet radiation and the shield. The glow may provide a visible indication that ultraviolet radiation is being intercepted by the shield.

A handle 150 allows the device to be moved easily. While the handle 150 is shown mounted on the upper portion 110, the handle 150 could also be mounted as easily on the base portion 120. The base portion 120 supports the upper portion and contains several operational and safety components. Wheels 190 mounted on the based portion 120 allow for easy maneuverability for the device 100. A display panel 160 presents operational information on a user interface and may receive user input such as an access code. The display panel 160 could be implemented as a touch-sensitive display device in order to both receive input from the user and to provide information about the device 100. Requiring the input of an access code prevents unauthorized use of the device 100, and therefore significantly improves the safety of a device in a medical situation where untrained patients might be able to gain access to the device.

A timer 170 is also found on the base portion 120. The timer 170 is a key safety and control feature that governs the activation and duration of the UV lamps. In one embodiment, the timer is configured with a delayed start function that allows the operator to initiate a disinfection cycle and then leave the area before the UV lamps energize. For example, a 90-second countdown can provide sufficient time for the operator to exit the room, close doors, or otherwise ensure the area is secure before germicidal light is emitted. This feature prevents the operator or bystanders from being inadvertently exposed to high-intensity UV radiation at the moment of activation, when the lamps may be operating at their peak output.

In addition to the delayed start interval, the timer 170 may include cycle duration control. The operator can select a sterilization period—such as 5 minutes, 15 minutes, or another programmed duration—after which the lamps automatically shut off. This limits unnecessary exposure time, conserves energy, and extends the service life of the bulbs that serve as the ultraviolet light source. More advanced embodiments may include programmable sequences, in which the UV lamps alternate between on and off intervals over a set cycle. Such cycles can be tailored to match disinfection requirements for specific pathogens, or to allow the device to operate over longer total periods without overheating or overexposing surrounding materials.

The timer 170 may also integrate with the device’s other safety systems, such that the countdown or cycle automatically resets or pauses if a motion sensor 180 detects movement that triggers a safety shutdown. In such a configuration, the device will only resume operation once it has verified that the area is safe. The timer 170 is shown separately from the display panel 160 in FIG. 1 in order to emphasize the importance of the timer 170. In an actual embodiment, it is expected that the timer 170 could be implemented digitally and accessed via the display panel 160.

The motion sensors 180 are designed to detect movement in the vicinity of the device and to respond in a manner that protects occupants from unsafe exposure to UV light. These motion sensors 180 may be positioned at multiple points on the device, such as on the front face of the base portion 120, near the upper portion 110, or integrated into the edges of the shield walls 130 and 140. Multiple motion sensors 180 can be used in combination to provide overlapping coverage zones, ensuring that approaching movement is detected from any direction.

In some embodiments, the motion sensors 180 operate with zonal detection capabilities, allowing the device to respond differently depending on where movement is detected. A primary detection zone may be configured to trigger an immediate lamp shutdown when movement is sensed on the unshielded side of the device. A secondary zone, positioned behind the shield walls, may have a different sensitivity threshold, allowing minor movement within the protected shadow area without interrupting the sterilization cycle. This distinction is important in clinical environments where a patient may be present in the shadowed zone, moving slightly without risk of exposure.

To support this differential response, one embodiment locates position sensors mounted on or in connection with the shield walls 130, 140, such as on the hinges of mounting arms that control the shield walls 130, 140 (as described below). These hinge-mounted position sensors detect the angular position of the left shield wall 130 and right shield wall 140 at each of their hinged joints, including the hinge connection to the upper portion 110, the mid-arm hinge, and the hinge at the connection to the shield wall. By reading the position of each hinge, the control system determines the precise angular extent and position of the shadow area created by the shield walls.

With the shadow area mapped in real time, the control system applies different response policies. Movement entirely within the shadow area is treated as safe and does not trigger a warning or shutdown, allowing a patient to shift position or adjust bedding without interruption. Movement detected near the edge of the shadow area—indicating that a person may be moving toward the exposed region—triggers a warning signal, which may be audible, visual, or both, to alert the person before they enter an unsafe zone. As used herein, an exposed region is a region that is not within a shadow region and that is expected to receive direct or significantly unattenuated ultraviolet light from the ultraviolet light source during operation. Movement detected in the exposed area, outside the shadow region, causes the UV lamps to shut off immediately, preventing direct exposure even if the person moves quickly into the beam. By integrating hinge-based shield position sensing with zoned motion detection and tiered response policies—safe, warning, and immediate shutdown—the device can safely provide UV sterilization in partially occupied rooms while maintaining a high level of occupant protection.

In an alternative embodiment, the device 10 determines, in situ, where germicidal UV light is actually being emitted and where it is blocked by the shield walls 130, 140. In one embodiment, a plurality of UV intensity sensors 182 are mounted around the circumference of the device, for example at evenly spaced azimuthal positions on or near the upper portion 110. In FIG. 1, these plurality of UV intensity sensors 182 are mounted at one vertical location, but such UV intensity sensors 182 can be mounted at two or more vertical elevations. Each UV sensor 182 is configured to detect germicidal wavelengths produced by the UV lamps and may include a UVC-sensitive photodiode with an optical filter, a protective window, and baffling to narrow its field of view to a defined sector.

Prior to full-power operation, a control system (not shown) that controls the device 100, including the display panel 160 and timer 170, and which monitors the motion sensors 180 and plurality of UV intensity sensors 182, performs a brief calibration sequence to generate an emission map. During this sequence, the UV lamps are energized at a reduced power level or in short pulses using the delayed-start window of the timer 170 so that no person is exposed to hazardous radiation. Each UV sensor 182 reports measured intensity for its sector; readings above a threshold indicate exposed sectors, while readings below the threshold indicate sectors occluded by the shield walls (shadow sectors). The control system applies hysteresis and noise filtering to the sensor data and then resolves the readings into a sector-by-sector emission map that identifies the angular extent and position of the shadow region relative to the device. When multiple vertical elevations are instrumented, the control system can also determine the vertical extent of the shadow region and adapt response logic based on expected occupant height (e.g., bed height).

The device then categorizes movement detected by the motion sensors 180 using this emission map. Movement occurring entirely within mapped shadow sectors is treated as safe and does not trigger a warning or shutdown. Movement detected with a trajectory toward the boundary of a shadow sector triggers a warning to alert the person prior to entering an exposed sector. Movement detected within mapped exposed sectors causes the UV lamps to shut off immediately. Because the emission map reflects where UV light is actually present or blocked, this zoned response remains valid even when the size and position of the shadow region changes due to shield repositioning.

In some embodiments, the control system repeats the calibration sequence whenever the device detects a change in shield position, after a prescribed elapsed time, or upon any discrepancy between expected and measured UV patterns. A discrepancy may be inferred when motion is detected in a sector classified as shadow while a UV sensor 182 simultaneously reports emission in that sector, in which case the device 100 fails safe by shutting off the lamps and prompting recalibration. To improve robustness, each sector’s threshold may be automatically adjusted over the life of the lamps to compensate for bulb aging and environmental factors such as ambient UV or reflections.

The foregoing mapping can be augmented or simplified depending on sensor choices. In one implementation, the UV intensity sensors 182 are co-located with the motion sensors 180 on shared boards around the device 100 so that each sector has a paired motion detector 180 and UV detector 182 with overlapping fields of view. The shared mechanical housing ensures co-registration between emission mapping and motion detection without software correction. The inner edges of the shield walls 130, 140 may optionally carry UV-absorbing or fluorescent coatings to increase contrast at the boundary between exposed and shadowed sectors, improving the reliability of boundary detection by nearby UV sensors.

In some embodiments the motion sensor 180 is also capable of determining the UV emission pattern. For example, a camera with a UV-pass filter can directly measure relative UV brightness across the field and produce an emission map while simultaneously tracking human motion. In such embodiments, separate plurality of UV intensity sensors 182 are not needed.

By deriving the emission map from measured UV intensity at the device rather than assuming idealized shield positions, the device continuously aligns its motion-response logic with the actual irradiance pattern present in the room. This approach allows safe, warning, and immediate-shutdown policies to be applied accurately even as the shield walls are repositioned to enlarge, shrink, or relocate the shadow region during use.

FIG. 2 shows a top view of the device 100 in the closed configuration. Venting holes 200 are visible on the top surface of the upper portion 110, allowing heat to escape and preventing overheating of internal components. A ventilation fan (not shown) can force air past any hot components in the device 100, such as the UV bulbs, and expel the hot air through the venting holds 200. The handle 150 is also visible in this view. With the shield walls 130 and 140 closed against the upper portion 110, no direct UV light can escape, allowing the device to be safely transported, stored, or prepared for use without risk of harmful exposure.

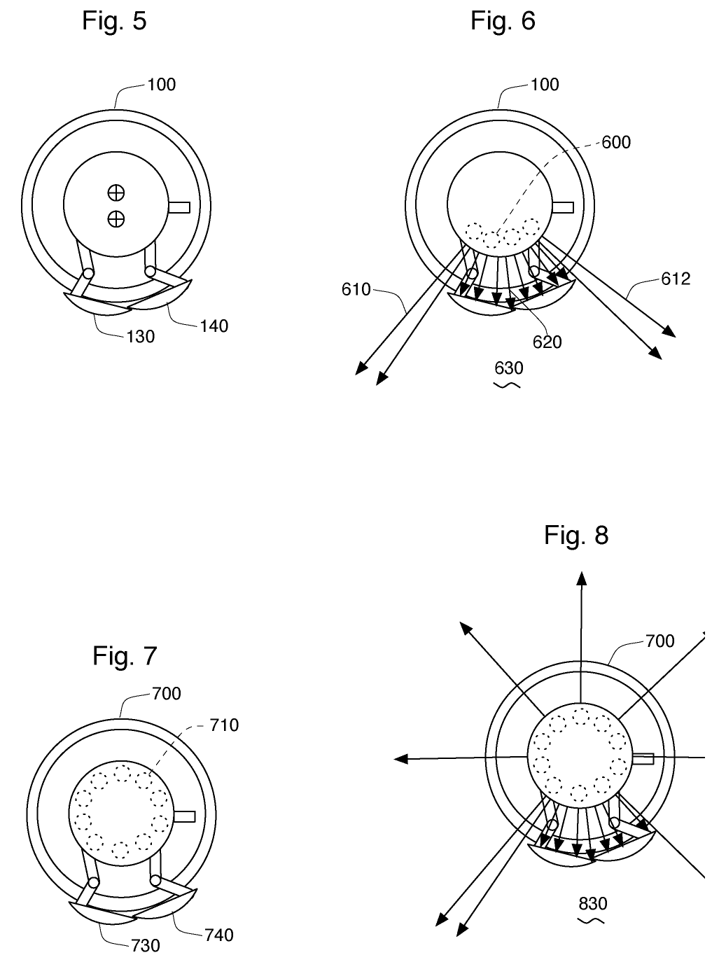

FIGS. 4 and 5 illustrate that the left shield wall 130 and the right shield wall 140 are positionable away from the upper portion 110 by way of left mounting arms 330 and right mounting arms 340. Two arms 330, 340 are shown for each shield wall 130, 140, with one of the arms 330, 340 located adjacent the upper edge of the walls 130, 140, and one adjacent the lower edge. The utilization of two arms 330, 340 for each wall 130, 140 helps to provide stiffness and to maintain the wall’s orientation as it is swung outward. In other implementations, a single arm 330, 340 may be used for a smaller or lighter walls 130, 140. Each mounting arm 330, 340 includes three hinged joints: a first hinge where the arm 330, 340 connects to the upper portion 110, a second hinge located intermediate along the arm 330, 340, and a third hinge where the arm 330, 340 connects to the corresponding shield wall 130, 140. These three hinges provide multiple rotational degrees of freedom so that each shield wall 130, 140 can be positioned at a selected angle and offset relative to the device 100 to shape the resulting shadow region that is screened from the ultraviolet light source by the shield walls 130, 140.

As explained above, position sensors can be mounted at the hinges to provide real-time measurement of the angular state of each mounting arm and wall. Mechanical features (such as detents, over-travel stops, or friction-adjustable pivots) may be included at one or more of the three hinges to help the operator place and retain each wall at the desired angle during use, while still allowing the hinge sensors to report position continuously for accurate mapping and safety control. This combination of multi-hinged arms and hinge-position sensing enables precise, independent placement of shield walls 130 and 140 as shown in the figures, while ensuring that motion-response logic remains synchronized with the actual shielding configuration.

The left shield wall 130 and the right shield wall 140 can be manipulated independently. An operator may open one wall while leaving the other closed, open both to different angles, or adjust the vertical arms asymmetrically to slant a wall 130 or 140 slightly for finer control of the shielded sector. This independent manipulation allows the shadow area to be enlarged, reduced, or reoriented to protect a patient, a pathway, or equipment while permitting disinfection elsewhere in the room, as the top-view configurations in FIGS. 4 and 5 depict.

FIGS. 5 and 6 present top views of device 100 with the left shield wall 130 and right shield wall 140 opened to establish a controlled shadow sector. FIG. 6 is the first view that depicts the UV bulbs 600, which comprises the UV light source for the device 100. In representative embodiments the bulbs are germicidal UVC lamps arranged as linear tubes mounted within the upper portion 110. Tube lamps may be positioned vertically around a cylindrical housing as previously described, and different bulb types and sizes may be used depending on the intended application and cycle time. In one practical example, the device employs industry-standard linear tubes of approximately four feet in length. Using such lamps allows for straightforward replacement via conventional end sockets accessible through a service opening, and the lamps may be protected by sleeves and retained with quick-change clips to simplify maintenance. When four-foot tubes are used, the upper portion 110 may be at least four feet high to accommodate the lamp length and end fittings while maintaining clearance for ventilation and wiring. Other lamp lengths and counts can be substituted without departing from the design. FIG. 6 further illustrates that the bulbs 600 can be installed only along a portion of the circumference behind the opened shield walls so that emission is concentrated in selected directions rather than 360° around the device.

With the bulbs 600 energized and the shield walls 130, 140 positioned as shown in FIG. 5, some UV rays 610 pass to the left of the left shield wall 130 and other UV rays 612 pass to the right of the right shield wall 140, while rays 620 are intercepted by one or both of the shield walls. This is shown in FIG. 6. The intercepted rays 620 define a shadow area or sector 630 that remains protected from direct UV exposure. The angular bounds of this shadow sector are set by the instantaneous positions of the shield walls and the adjacent escaping ray paths 610 and 612 as shown in the figures.

This device 100 can be used to disinfect different clinical areas in hospitals, clinics as well as other public spaces. The machine 100 will allow the operator to disinfect the surfaces as well as the air next to a highly infectious patient or persons with UV light. The apparatus will be able to disinfect the air, which will help in the case of an airborne illness, like Covid, Tuberculosis, etc. The advantage of this configuration is that persons can be next to the unit safely while it is in use. This is more advantageous in the clinical setting as existing, prior art machines of this type require everyone to leave the room. There is not much time when people are not present in the medical field, therefore, to have a device that can disinfect the room with some people present is essential. The disclosed apparatus 100 may be used in a room with a Methicillin resistant pathogen, Clostridium Difficile, etc. A staff member can enter the room before use to disinfect surfaces to help curb nosocomial infections. After this, the device is left in the room and turned on while the patient remains in the room, protected from exposure by the shield walls 130, 140.

In some implementations, the device may be accompanied by instructions for use that direct an operator to identify reflective surfaces in the room before initiating a UV disinfection cycle. Reflective surfaces may include, for example, mirrors, metallic or silver-colored picture frames, polished metal surfaces, glass surfaces, or other objects capable of reflecting ultraviolet light. The instructions may direct the operator to remove, reposition, or cover such reflective surfaces before operation. The covering may include a sheet, drape, cloth, disposable cover, UV-attenuating material, or other material positioned over the reflective surface to reduce unintended reflection of ultraviolet light during the disinfection cycle.

### Alternative Embodiments

FIGS. 7 and 8 illustrate a second embodiment 700 in which UV bulbs 710 are arranged around the entire circumference of the upper portion 110. The left shield wall 130 and the right shield wall 140 are opened in the same general positions as in FIGS. 5 and 6, but the exposure pattern differs because the circumferential bulbs emit in all outward directions when unblocked. This configuration enables substantially uniform irradiation of the room except where the shield walls intercept the rays.

FIG. 8 depicts the resulting ray pattern. Rays propagate outward in multiple directions from the circumferential bulbs 710, while the opened shield walls 730 and 740 define a shadow sector 830. The angular size of shadow sector 830 is similar to shadow sector 630 described with respect to FIG. 6, but here the remainder of the room outside sector 830 is exposed due to emission from the full circumference of bulbs 710. The boundary of the shadow sector is set by the edges of the shield walls and the adjacent rays escaping on either side.

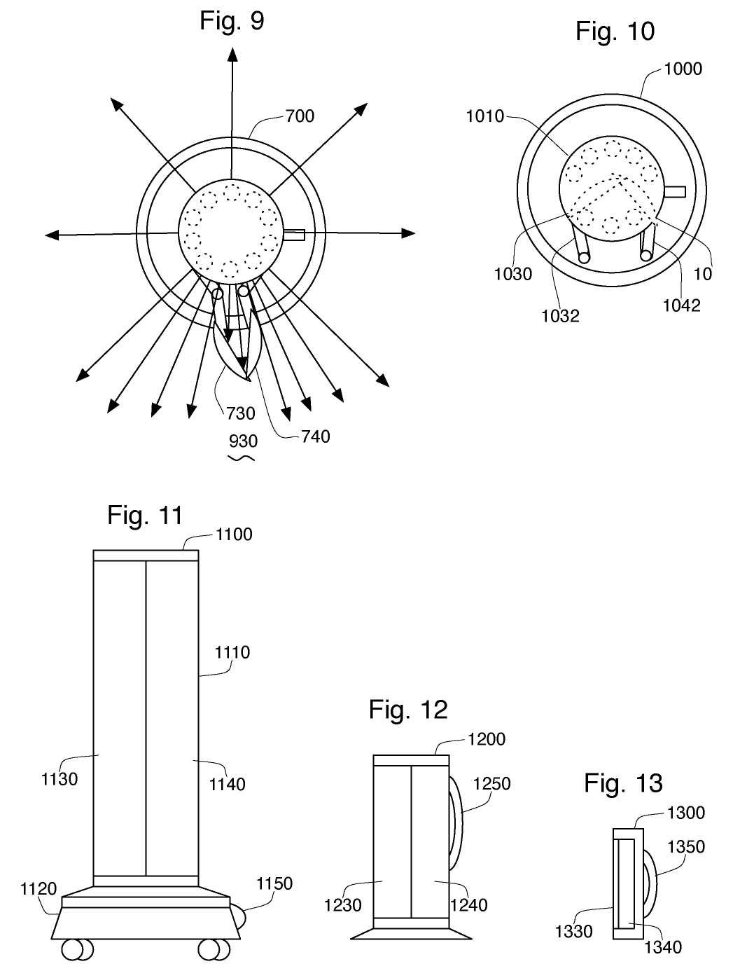

FIG. 9 shows a further manipulation of the shield walls to decrease the size of the protected region. By rotating the left shield wall 730 and right shield wall 740 toward one another and adjusting their angles with the mounting arms, the device 100 narrows the blocked region and produces a smaller shadow sector 930. This arrangement demonstrates that the device 100 can finely tune the angular width and orientation of the protected zone 930 while maintaining broad exposure elsewhere in the room.

FIG. 10 shows a third embodiment in which a device 1000 includes an upper section 1010 configured with internal receiving slots (not shown in FIG. 10) that allow the left and right shield walls 1030 and 1040 to be retracted into the interior of the upper section 1010. The shield walls 1030, 1040 are still coupled to the device by hinged arms 1032 and 1042 so they can be deployed and manipulated in the same manner as described for the earlier embodiments, and fully retracted when unobstructed emission is desired.

The receiving slots are positioned so that, when the arms 1032, 1042 are folded at their mid-hinges and rotated toward the upper section 1010, each shield wall aligns with and slides into a corresponding slot. In one implementation, the slots are vertical channels opening at the lower rim of the upper section and extending upward along the interior surface to a depth sufficient to nest the entire shield wall thickness. In another implementation, the slots open radially through the sidewall at discrete azimuthal locations and lead into an annular internal cavity; the slot mouths may be slightly flared to guide the walls during insertion. Guide rails, low-friction liners, or detents may be included within the channels to stabilize the walls in their stowed positions and to prevent rattle during transport.

To accommodate retraction without contacting the UV lamps, the bulb layout in the upper section is arranged with keep-out corridors aligned to the slot paths. For example, circumferential bulb arrays may omit lamps directly behind each slot or use shortened lamps on either side of the corridor, leaving a clearance gap through which the shield walls 1030, 1040 pass. Alternatively, the lamps may be mounted on segmented rings that terminate before the slot locations, with reflectors bridging the gap to maintain uniform output elsewhere. These corridors can be sized to account for wall thickness, arm hardware, and installation tolerances.

During use, the operator may withdraw one or both shield walls from their slots and articulate them via arms 1032 and 1042 to define a desired shadow sector, as in the first and second embodiments. When both walls 1030, 1040 are fully retracted, the upper section 1010 presents an unobstructed 360-degree emission pattern (subject to the keep-out corridors). The same hinge-position sensors used in the earlier embodiments can be employed on the device-side hinge, mid-arm hinge, and wall-side hinge to report whether a wall is stowed within a slot or deployed, allowing the control system to update the emission map accordingly and apply the movement response policies described elsewhere (safe within shadow, warning at the boundary, immediate shutoff in exposed sectors).

FIG. 11 illustrates embodiment 1100 in which the base portion 1120 is minimized so that the device is predominantly the upper portion 1110. A low-mounted handle 1150 provides leverage close to the floor for rolling, docking, or pulling the device, while the tall upper portion 1110 increases the available lamp height for room-scale disinfection within a compact footprint. This configuration is advantageous in tight clinical environments—such as crowded patient rooms or corridors—because the smaller base 1120 eases maneuvering around beds and equipment while preserving lamp column height for effective irradiation. Shield walls 1130 and 1140 remain deployable and adjustable via the mounting arms to define a selected shadow sector as described above.

FIG. 12 shows embodiment 1200, a tabletop implementation intended for portable, localized disinfection rather than whole-room coverage. The reduced overall height and integrated pedestal facilitate placement on work surfaces, carts, or bedside tables to disinfect nearby air and exposed surfaces—useful for procedure trays, small exam rooms, or shared equipment between patients. Shield walls 1230 and 1240 remain present and can be manipulated to protect an adjacent occupant or pathway while allowing exposure elsewhere on the table or counter. A rear handle 1250 assists hand-carry and repositioning between use sites.

FIG. 13 presents embodiment 1300, a handheld implementation for operator-directed disinfection of small areas, fixtures, or equipment. A rear handle 1350 permits one-handed use while the shield structure 1330 and 1340 is configured to prioritize protection of the user. In some implementations, the travel of the shield walls is limited so that, when the device is gripped by the handle, the shields can be positioned only to block UV emission toward the operator. In this handheld mode, the shields will not be used to create a remote shadow sector in the room. Instead, they provide localized, user-facing protection while permitting forward emission toward a target surface. In some embodiments, the handheld device 1300 may also be placed on a table, cart, counter, floor, or other support surface and operated through a timed disinfection cycle without being held by the operator.

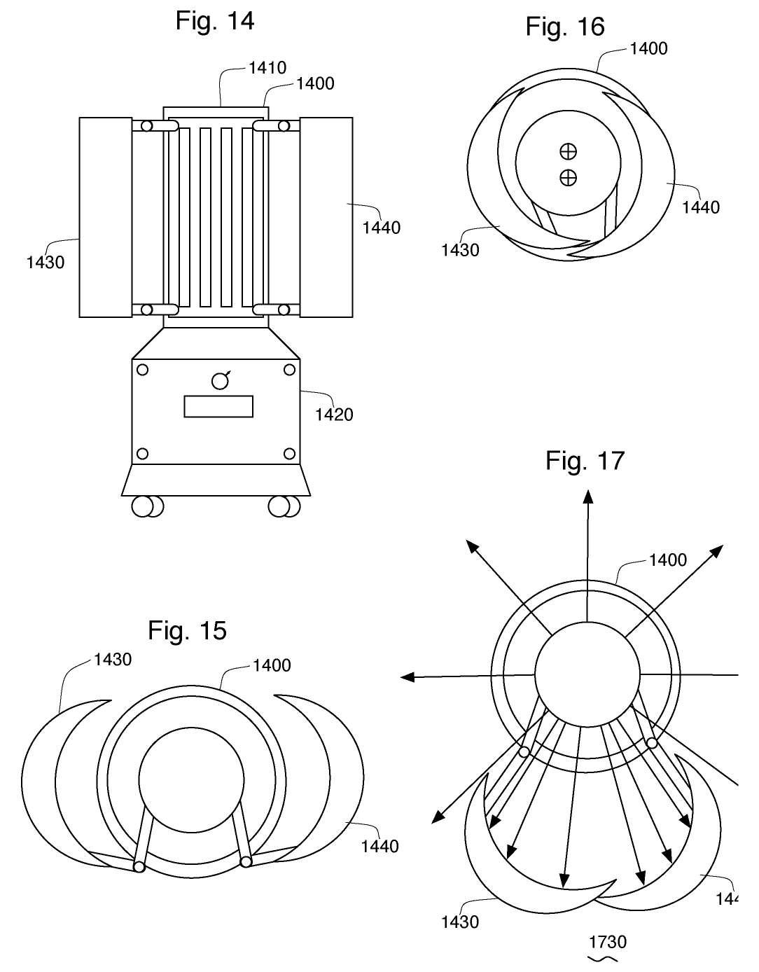

FIGS. 14-17 illustrate device 1400, which includes an upper portion 1410 and a lower portion 1420. In this embodiment the left shield wall 1430 and the right shield wall 1440 are substantially larger than in earlier figures and are shaped as large arcs. Such arc-shaped walls may be formed by roughly parallel inner and outer walls, or in a crescent shape as shown in the Figures. By way of example, the upper portion 1410 may have a diameter of about eighteen inches, while each shield wall 1430, 1440 may has an end-to-end span (straight-line distance between distal ends) of about three feet. In one embodiment, the upper portion has a diameter in a range of about 14–22 inches and each shield wall has an end-to-end span is in a range of about 30–42 inches. Owing to their increased curvature and size, the shield walls 1430, 1440 do not wrap directly against the outer surface of the upper portion 1410 when retracted, as shown in FIG. 16. In other words, the shield walls 1430, 1440 remain spaced from and do not conformingly wrap against an outer surface of the upper portion, in contrast to the embodiment shown in FIGS. 1-4.

FIGS. 14 and 15 show the large shield walls 1430, 1440 partially extended. In this position, the device may be energized so that the walls intercept portions of the emitted UV light and create two substantial shadow areas—one generally forward of each wall. This two-sector arrangement is useful when two protected regions are desired simultaneously, for example shielding a patient bed and an adjacent pathway, or shielding a caregiver work zone while also protecting a doorway from direct exposure. The large arcuate geometry increases the angular span and depth of each shadow, giving occupants more freedom to move safely within those protected regions while the remainder of the room continues to receive germicidal exposure.

FIG. 17 shows the device energized with shield walls 1430 and 1440 brought toward each other to create a single, contiguous shadow area 1730. This configuration is advantageous when a broader protected zone is needed—such as for temporary staff presence, equipment staging, or moving a patient—while still allowing disinfection of the rest of the room. The size and placement of the large shadow area 1730 are set by the angular positions of the two walls and can be reoriented as needed to protect a particular region while preserving exposure elsewhere.

As with the earlier embodiments, each shield wall 1430 and 1440 can be manipulated independently. Tilting one wall relative to the other—by adjusting the angles provided by the arm hinges—narrows the resulting shadow on that side and shifts the boundaries of the protected zone. This selective canting allows the operator to fine-tune the shadow width to match a smaller target (for example, a narrow passage or specific instrument cart) while maintaining the larger shadow from the opposite wall or combining both for the large area 1730 when needed. Hinge-position sensing can be used to map these wall positions in real time so that the control system applies the appropriate safe/warn/shut-off policies described earlier.

Each of the disclosed embodiments can also be useful in settings where staffing is limited, helping to ensure that a treatment area is reliably disinfected even when personnel are not available to oversee a conventional cleaning. Because the shielding defines shadow regions in which occupants and protected items may remain during a treatment cycle, the disclosed devices allow germicidal treatment to be carried out consistently and with reduced reliance on dedicated staff.

### Shield Wall Attachment and Movement

The shield walls 130 and 140 may be attached to the device in numerous alternative ways. In addition to the multi-hinged arms described above, suitable attachments include single or multiple hinges (including “living” hinges formed in a compliant member), fixed mounts, sliding-rail carriages, and snap-fit couplings. The walls may be secured by screws and bolts, by magnetic latches, by straps or cables, by adhesive bonding, or by interlocking profiles such as dovetail slots (optionally with a counterweight to assist movement). A collar mounted around the upper portion may cooperate with a wedge element to clamp or index the wall position. Lead-screw and wedge mechanisms can provide fine adjustment in both vertical and horizontal directions. Any two or more of these attachment methods may be combined.

Various motion mechanisms may be employed to deploy or retract the walls 130, 140. Examples include rotating cams, cable-and-pulley drives, sliding rails with a movable carriage, spring-assisted linkages, and electrically driven actuators such as solenoids or motors. Motors may be rotary or linear actuators; they may be powered by line electricity or by an onboard battery. Electromagnetic activation may be used to release, latch, or drive the walls. These mechanisms can be integrated with the position sensing and control features described elsewhere, but the particular attachment and drive choices are not limited to the examples above.

In one embodiment, the shield walls 130 and 140 are positioned manually by the operator using the three-hinged mounting arms (left mounting arms 330 and right mounting arms 340). Friction-adjustable pivots, detents, or over-travel stops may be provided at one or more hinges so the walls 130, 140 hold a selected angle during use yet can be quickly folded to a stowed position for transport.

In automated embodiments, the three-hinged mounting arms 330, 340 are driven by motors or linear actuators coupled to one or more hinges. The display panel 160 provides controls to extend, retract, and angle the walls. The panel can present directional soft-keys or a joystick-style control to incrementally open or close each wall, as well as a “retract” command that folds both walls 130, 140 to a transport position. Position sensors mounted on the hinges of the mounting arms 330, 340 can provide feedback for the controller in order to properly position the walls 130, 140.

The device 100 may store pre-set positions—for example “doorway shield,” “bedside shield,” “narrow aisle,” and “fully closed.” When a preset is selected on the display panel 160, the controller energizes the actuators to move the walls 130 and 140 to the corresponding angles and offsets that were stored in that preset. Hinge-position sensors report actual angles during motion so the controller can synchronize both sides, smooth acceleration and braking, and stop precisely at the target geometry.

For fine adjustments, the display panel 160 can offer nudge controls to change a wall by small angular increments, allowing the operator to tighten or widen the shadow sector without stopping the ongoing cycle. A dedicated retract control on the panel 160 may fold both walls to a compact profile for moving the device between rooms, and an equalize control may symmetrically align the walls when desired. In other words, the display panel 160 will present control options to the user, and accept the selection of those control options, allowing the user to select a stored preset of wall positions and angles, to perform incremental nudge adjustments, to instruct the device 100 to retract the walls 130, 140 in order to prepare the device 100 for transport, and to equalize or symmetrically align the first and second shield walls.

Safety interlocks may inhibit powered motion unless the controller has verified that the area is safe and may reduce actuator speed when people are present. During any automated movement, the controller continues to monitor the hinge-position sensors and motion sensors. If an obstruction is detected or the current draw exceeds a limit, motion halts and an alert is issued. An alert is also issued if any other type of unexpected stall in the movement of the walls 130, 140 is detected. Manual operation remains available as a fallback, as the operator can disengage the drives or use a clutch release to reposition the walls by hand, after which the controller updates the stored wall positions from the hinge sensors.

### Controller

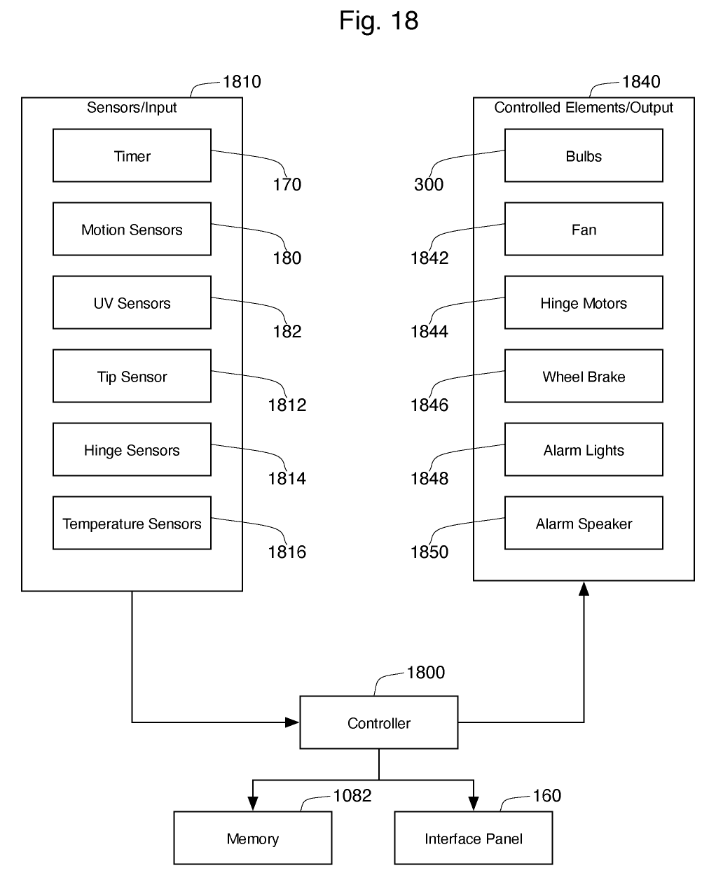

FIG. 18 shows a controller 1800 that controls the functioning of the device 100. The controller 1800 includes one or more hardware processors operatively coupled to memory 1082 and to the device’s sensors 1810 and actuators 1840 via electrical signal lines and data buses. In representative embodiments the controller 1800 is a microcontroller or microprocessor-based module mounted on a printed circuit board within the housing. The controller 1800 executes program instructions to coordinate operation of the bulbs 300, interpret data from the sensors 1810, position the shield walls 130, 140, and implement safety interlocks and user-selected modes.

Program code for the controller is stored on a non-transitory computer-readable medium such as memory 1082, which may include any combination of flash, EEPROM, and RAM. Memory 1082 can further store configuration parameters, calibration constants, look-up tables (e.g., sector maps and threshold values), usage logs, and pre-set wall positions. On power-up the controller loads the program from memory 1082 and initializes peripherals, timers, and I/O.

During operation, the controller 1800 periodically samples the connected sensors 1810, including the motion sensors 180, the UV sensors 182, hinge position sensors 1814, temperature sensors 1816, and a tip sensor 1812. Based on those inputs, and on user selections received from the display panel 160 and timing information from the timer 170, the controller 1800 generates control outputs to drive the bulbs 300, a fan 1842, hinge motors 1844 that position the shield walls, a wheel brake 1846, alarm lights 1848, and an alarm speaker 1850. The controller may implement a real-time clock and software timers using parameters from the timer 170 to provide delayed start and cycle timing, and may maintain a watchdog routine to return the system to a safe state if the program becomes unresponsive. The wheel brake 1846 can be automatically engaged whenever the controller 1800 initiates a sequence in which the UV light source is illuminated to prevent the device 100 from moving unexpectedly.

In automated embodiments the controller 1800 executes routines that translate desired shield-wall positions into actuator commands for the hinge motors 1844, monitors the hinge sensors 1814 for closed-loop feedback, and adjusts speed and acceleration profiles to achieve smooth motion with positional accuracy. In safety-critical routines, the controller classifies detected movement from the motion sensors 180 by comparing those signals to an emission or shadow map derived from the UV sensors 182 and the hinge sensors 1814, and then applies the appropriate policy. The policy may permit the detected movement, issue a boundary warning via the alarm lights 1848 and alarm speaker 1850, or shut off the bulbs 300. Thermal management uses the temperature sensors 1816 to modulate the fan 1842 and to de-energize the bulbs 300 on over-temperature, while the tip sensor 1812 triggers a shutoff if the device is knocked over. All such functions are performed by the hardware processor of the controller 1800 executing software stored in memory 1082 on a non-transitory computer-readable medium, thereby providing a concrete implementation of the described control logic.

The controller 1800 is responsible for determine the shadow region cast by one or more of the shield walls. In the embodiment where this is performed based on sensors in the mounting arms 330, 340, the controller 1800 establishes a device-fixed reference azimuth of 0 degrees aligned to a known feature on the housing (for example, the mid-point of the display panel 160). All angular values are expressed in degrees 0–360 relative to this reference, increasing in a defined direction (e.g., clockwise when viewed from above).

Using signals from the hinge position sensors 1814, the controller 1800 computes boundary angles corresponding to the edges of the shadow cast by the first shield wall 130 and, when present, the second shield wall 140. From these boundary angles the controller 1800 defines an angular location of the shadow region as a range between two boundary angles referenced to the device-fixed 0-degree direction. By way of example, an angular location may be defined as 82 degrees to 105 degrees. If the boundary angles straddle 0 degrees, the range wraps accordingly (for example, 320 degrees to 20 degrees). The controller 1800 updates the angular location of the shadow region continuously as the shield walls 130, 140 move.

In one implementation, the controller resolves each shield wall’s in-plane orientation from the sensed angles at the device-side hinge, intermediate hinge, and wall-side hinge, and then determines the wall edge orientations that bound the shadow. Those edge orientations establish the two boundary angles that define the angular location of the shadow region. The controller may optionally apply a small safety margin to one or both boundary angles to account for wall thickness, sensor tolerance, or expected reflections.

The angular location so defined is used to categorize movement detected by the plurality of motion sensors 180. Motion that lies within the two-angle angular location is treated as movement in the shadow region and does not cause UV light source to shutoff. Motion whose azimuth lies outside the range is treated as movement in the exposed region and causes the controller to deactivate the UV light source. Motion whose azimuth lies near either boundary angle (within a configurable tolerance) may trigger a warning via the alarm lights 1848 and/or alarm speaker 1850 while the light source remains energized.

In still other embodiments, the apparatus may be implemented without a computerized controller, or without any controller that performs automated control functions. In such embodiments, the shield walls may be manually moved, positioned, and secured by an operator before initiating a disinfection cycle, without the use of automated arms, hinge motors, computer control, or software-based positioning logic. The ultraviolet light source may be activated and deactivated using manual switches, mechanical timers, relays, hardwired interlocks, or other non-computerized control components. In some embodiments, the apparatus may rely primarily or entirely on manually adjustable mechanical structures, such as hinges, friction pivots, detents, latches, clamps, or slides, to position and hold the shield walls in selected locations. Thus, although controller-based embodiments are described above, the apparatus may also be implemented as a simplified manually operated device in which shield positioning and ultraviolet activation are controlled manually rather than by a computerized controller.

### Additional Embodiments

As shown in FIG. 1, the device 100 may further include a remote control 104 operatively coupled to the controller 1800 by a wired or wireless link and configured to provide off-device user interaction for safety and convenience. In use, the remote control 104 allows an operator to arm the timer 170 and initiate a delayed-start countdown from outside the room or behind a barrier, to issue an immediate stop command that de-energizes the bulbs 300, and to pause or resume an ongoing cycle without reprogramming. The remote control 104 may present selection of stored preset positions for the first and second shield walls, provide incremental nudge adjustments, and issue a retract-to-transport command to fold the walls prior to moving the device 100. Audible and visual alerts may be acknowledged from the remote, including silencing the alarm speaker 1850 while maintaining the alarm lights 1848, and a “find unit” function may flash the alarm lights 1848 to locate the device in storage. Live status—such as countdown, UV-on state, remaining cycle time, brake status, and fault indicators—can be displayed on the remote so staff can monitor progress without entering the exposure area. In certain implementations, the remote can trigger a calibration or mapping sequence, select a “doorway mode” that preemptively reduces output or reorients shields for anticipated ingress, and execute workflow macros that combine preset selection and countdown in a single action.

For security and process control, the remote control 104 may require entry of an authorization code to arm the timer 170 or enable UV output, may be range-limited so that commands are accepted only from a safe distance, and may be securely paired to a single device to prevent inadvertent cross-control. In some embodiments the remote can temporarily lock out the interface panel 160 during a sterile procedure, confirm engagement of the wheel brake 1846 before permitting lamp enable, and report maintenance and health information such as lamp hours, fan temperature, and sensor status. The remote can also coordinate staggered starts across multiple devices positioned in adjacent rooms or corridors, and may provide a positive “cycle complete” indication so personnel receive confirmation that the bulbs 300 are off prior to entry. In handheld synergy implementations, the remote control 104 may be worn or clipped to the operator and include a local UV sensor 182; when the remote detects irradiance above a threshold at the operator’s position, the controller 1800 inhibits or reduces lamp output to maintain user-facing protection during handheld use.

In another alternative embodiment, the shield structures are flat walls 130 and 140 rather than arcuate panels. Each wall is formed as a generally planar UV-attenuating panel (e.g., polycarbonate or acrylic) carried by a peripheral frame that couples to the mounting arms (e.g., the three-hinge arms described above). The flat walls 130, 140 pivot about their respective hinges so that an operator can position them independently to create one or more shadow regions. When a single wall is deployed, its planar edge defines a straight boundary of the shadow; when both are deployed, the walls can be canted to form a V-shape or U-shape that narrows or widens the protected sector. Because the edges are straight, the angular location used by the controller (when hinge sensing is employed) is obtained directly from the orientations of the two panel edges that face the light source, with wrap-around handling as previously described.

Flat walls can include features to improve shielding performance and usability. In some implementations, the panel edges include overlapping lips, beveled edges, or interlocking tongues/gaskets to reduce stray emission at the boundary when two panels are brought near one another. Stiffening ribs or a lightweight metal frame may be provided to maintain planarity, and detents or friction pivots at one or more hinges hold the walls at selected angles. Flat walls can also be adapted to the retractable slot embodiment: the panel thickness is sized to slide within vertical channels in the upper portion, with keep-out corridors among the lamps as previously described. Latching may be by magnetic catches, spring latches, or snap-fit features on the frame. This flat-panel configuration preserves the same operational modes as the arcuate versions—independent manipulation of left and right walls to form two separate protected regions, or bringing the walls together to form a single larger protected region—while offering simplified fabrication and straightforward serviceability.

In yet another alternative embodiment, the device 100 omits the shield walls and operates as a compact, whole-room ultraviolet sterilizer with enhanced safety controls. The housing includes the upper portion 110 that contains the ultraviolet light source (bulbs 300) and the base portion 120. A controller 1800 (with programming stored in memory 1082) coordinates operation of the bulbs 300 in response to inputs from a timer 170, motion sensors 180, UV sensors 182, a tip/tilt sensor 1812, and temperature sensors 1816. In typical use, an operator positions the device, engages the wheel brake 1846, arms the timer 170, and initiates a delayed-start countdown from the interface panel 160 or a remote control 104. During the countdown the bulbs remain de-energized and audible/visual indicators (alarm speaker 1850, alarm lights 1848) signal the impending cycle. If motion sensors 180 detect entry into the exposure area at any time, the controller immediately de-energizes the bulbs 300; if a tip/tilt or over-temperature condition is detected, the controller likewise shuts down the bulbs and drives the fan 1842 as needed to restore safe conditions.

Even without physical walls, this embodiment can provide improved safety and workflow. The UV sensors 182 may be used to verify irradiance before and during a cycle and to adapt lamp drive for bulb aging or room reflectivity; the remote control 104 may include a local UV sensor to inhibit or reduce output when excessive irradiance is detected at the operator’s position (e.g., during handheld use or while standing just outside a doorway). The timer 170 supports delayed start and cycle limits so rooms can be disinfected after staff exit, while the panel 160 (or remote 104) can pause and resume cycles to accommodate brief entries without reprogramming. Optional lamp zoning—implemented electrically by segmenting the bulb array into circumferential zones—allows selected sectors to be dimmed or disabled for workflow (e.g., a doorway mode) while leaving the remainder of the room under active disinfection. Event logging (e.g., start/stop times, interruptions, and faults) may be maintained in memory 1082 for compliance. This shield-less configuration therefore remains useful for rapid turnover disinfection, after-hours cycles, or applications where physical barriers are impractical, while retaining multiple, redundant controls that protect occupants from unintended UV exposure.

### UV Patient and Room Sensors

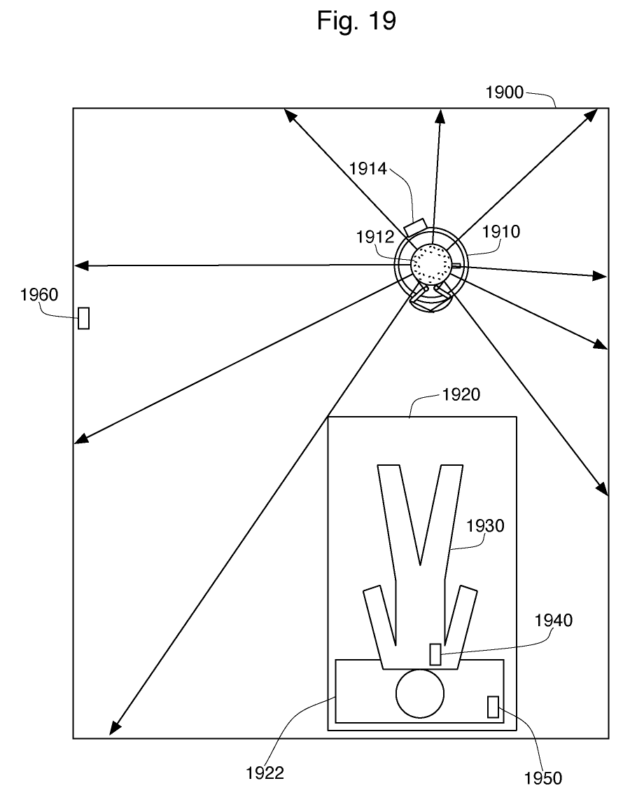

FIG. 19 illustrates an embodiment for monitoring ultraviolet exposure in a room 1900 during operation of a UV sterilization device 1910. The device 1910 includes a UV light source 1912 and a graphical user interface 1914, which may receive user input and present information relating to an active disinfection cycle. In some embodiments, the UV light source 1912 may comprise one or more UV lamps, UV LEDs, excimer lamps, UV laser sources, or other ultraviolet-emitting devices. When the UV light source 1912 comprises a UV laser source, the ultraviolet treatment device 1910 may include one or more optical elements configured to expand, diffuse, scan, reflect, refract, or otherwise distribute ultraviolet light from the UV laser source over a treatment area. For example, the optical elements may include a diffuser, lens, beam expander, scanning mirror, rotating mirror, reflector, light pipe, waveguide, or other optical structure configured to reduce localized intensity and provide a desired ultraviolet irradiance pattern. The controller may operate the UV laser source and associated optical elements to deliver ultraviolet radiation to the exposed region while monitoring motion sensors, ultraviolet sensors, remote ultraviolet sensors, and other safety inputs as described herein.

The room 1900 may include a bed 1920, a pillow 1922, and a patient 1930 located in or near an area intended to be protected from excessive ultraviolet exposure. Because ultraviolet exposure at the patient 1930 may vary depending on the position of the patient, bedding, shielding, and other objects in the room 1900, one or more UV sensors may be positioned at different monitored locations within the room 1900.

In the embodiment of FIG. 19, a personal UV sensor 1940 is associated with the patient 1930, a pillow sensor 1950 is positioned at or near the pillow 1922, and a room sensor 1960 is positioned at another location within the room 1900. The personal UV sensor 1940 and pillow sensor 1950 may be associated with a shadow region in which the patient 1930, pillow 1922, bed 1920, or other monitored location is intended to be protected from direct ultraviolet exposure. Accordingly, the sensors 1940 and 1950 may be used to detect unintended or excessive ultraviolet exposure within the shadow region. By contrast, the room sensor 1960 may be associated with an exposed region of the room 1900 in which ultraviolet exposure is intended during the disinfection cycle. The room sensor 1960 may therefore be used to measure the ultraviolet dose delivered to the exposed region, confirm that ultraviolet treatment occurred, or determine when a desired treatment dose has been reached. Each of the sensors 1940, 1950, and 1960 may communicate exposure information to the device 1910 for use during the disinfection cycle, but the exposure information from sensors associated with a shadow region may be evaluated differently than exposure information from a sensor associated with an exposed region. The graphical user interface 1914 may be used to associate one or more of the sensors 1940, 1950, and 1960 with the device 1910, the room 1900, the patient 1930, the shadow region, the exposed region, or the particular disinfection session before the UV light source 1912 is energized.

This association is useful in facilities where multiple UV sterilization devices and multiple UV sensors may be used. A sensor located in one room should not inadvertently control a device operating in a different room, while a sensor associated with a particular patient should be capable of being linked to whichever device is being used for the disinfection cycle in that patient’s room. Accordingly, the device 1910 may accept exposure information for safety-control purposes only from sensors associated with the active disinfection session.

The use of multiple sensor locations also addresses the possibility that one sensor may not accurately represent ultraviolet exposure at all relevant locations. For example, the personal UV sensor 1940 may be covered by bedding, blocked by clothing, turned inward, or otherwise occluded. The pillow sensor 1950 or the room sensor 1960 may provide additional exposure information from other locations in the room 1900. If a sensor is not communicating, is not associated with the active session, appears to be improperly positioned, or reports exposure data indicating that a permitted exposure limit is being approached or exceeded, the device 1910 may issue an alarm, update the graphical user interface 1914, reduce output of the UV light source 1912, or deactivate the UV light source 1912.

The UV sensors 1940, 1950, and 1960 may be implemented as ultraviolet sensors, ultraviolet dosimeters, digital dosimeters, Bluetooth-enabled dosimeters, wearable UV tags, room UV sensors, or other devices capable of measuring ultraviolet radiation at or near a monitored location. In some embodiments, one or more of the UV sensors 1940, 1950, and 1960 measure cumulative ultraviolet exposure over time, rather than merely detecting instantaneous ultraviolet intensity. For example, the personal UV sensor 1940 may be a patient-worn or patient-associated digital dosimeter that registers the cumulative amount of ultraviolet radiation received at or near the patient 1930. Commercially available UV dosimeters, including a UV/cense Digital Dosimeter or a Bluetooth dosimeter, may be used as examples of suitable devices, although other ultraviolet sensors or dosimeters may also be used.

Each UV sensor 1940, 1950, and 1960 may generate an output representing measured ultraviolet exposure. The output may indicate instantaneous ultraviolet intensity, cumulative ultraviolet dose, exposure time, threshold status, sensor status, or combinations thereof. In some embodiments, the UV sensor provides an analog or digital signal to the device 1910 or to an intermediate control component. For example, a sensor output may be received by a data acquisition device, a microcontroller, a programmable logic controller, a USB data acquisition module, or another interface that converts the sensor output into information usable by the device 1910. The interface may then communicate the sensor information to the device 1910 using a wired connection, wireless connection, USB connection, serial connection, Ethernet connection, Bluetooth connection, or other suitable communication path.

The device 1910 may compare the ultraviolet exposure data received from the UV sensors 1940, 1950, and 1960 with one or more predetermined exposure thresholds. If the exposure data indicates that ultraviolet exposure at a monitored location is below a threshold, the device 1910 may continue operation of the UV light source 1912. If the exposure data indicates that ultraviolet exposure is approaching, has reached, or has exceeded a threshold, the device 1910 may update the graphical user interface 1914, issue an audible or visual alarm, reduce power to the UV light source 1912, pause the disinfection cycle, or deactivate the UV light source 1912. In some embodiments, a hardwired safety relay or physical emergency stop switch may be provided so that the UV light source 1912 can be deactivated independently of software-based control.

The room sensor 1960 may be positioned at a location in the room 1900 that is intended to receive ultraviolet radiation during a disinfection cycle. Unlike the personal UV sensor 1940, which may be positioned to monitor exposure at or near the patient 1930, the room sensor 1960 may be purposefully exposed to the ultraviolet light emitted by the UV light source 1912 so that the device 1910 can determine whether an appropriate ultraviolet dose has been delivered to the room 1900 or to a selected portion of the room 1900. The room sensor 1960 may therefore be used to confirm that the disinfection cycle actually occurred, to measure cumulative ultraviolet exposure received at the room sensor location, and to determine whether a desired room-treatment dose has been reached. Data from the room sensor 1960 may also be stored as part of a treatment record for the room 1900, including the date and time of treatment, the cycle duration, the cumulative dose received, and whether the cycle was completed or interrupted. In some embodiments, the room sensor 1960 may also be used to verify operation of the UV light source 1912, detect reduced lamp output, confirm that the room sensor 1960 is associated with the correct room or treatment session, provide a reference measurement for comparison with the personal UV sensor 1940 or pillow sensor 1950, and assist in determining whether the device 1910 should continue operation, issue an alarm, update the graphical user interface 1914, or deactivate the UV light source 1912.

In some embodiments, the device 1910 records cumulative ultraviolet exposure associated with a patient 1930 over a plurality of treatment cycles. During a current treatment cycle, the device 1910 may receive ultraviolet exposure data from a single patient-associated sensor, such as the personal UV sensor 1940, or from multiple sensors associated with the patient 1930 or with the shadow region, such as the personal UV sensor 1940 and the pillow sensor 1950. The device 1910 may use the ultraviolet exposure data received during the current treatment cycle to update a patient cumulative exposure record that includes ultraviolet exposure accumulated during previous treatment cycles for the same patient. When multiple sensors are used during a treatment cycle, the device 1910 may store the exposure data from each sensor separately, may determine a representative patient exposure value from the multiple sensors, or may determine a cumulative patient exposure value using data from the multiple sensors before updating the patient cumulative exposure record. The device 1910 may compare the updated patient cumulative exposure record to a permitted exposure limit and may issue an alarm, reduce ultraviolet output, or deactivate the ultraviolet light source 1912 when the updated patient cumulative exposure record reaches the permitted exposure limit. The patient cumulative exposure record may be associated with patient identification information entered through the graphical user interface 1914 before the treatment cycle.

The particular number, type, and arrangement of UV sensors shown in FIG. 19 are exemplary. In other embodiments, the system may use only one of the sensors 1940, 1950, and 1960, any two of the sensors 1940, 1950, and 1960, or additional sensors positioned at other locations in the room 1900. The sensors may be patient-worn, patient-adjacent, bed-mounted, pillow-mounted, wall-mounted, device-mounted, room-mounted, or otherwise positioned to monitor ultraviolet exposure at one or more selected locations during a disinfection cycle.

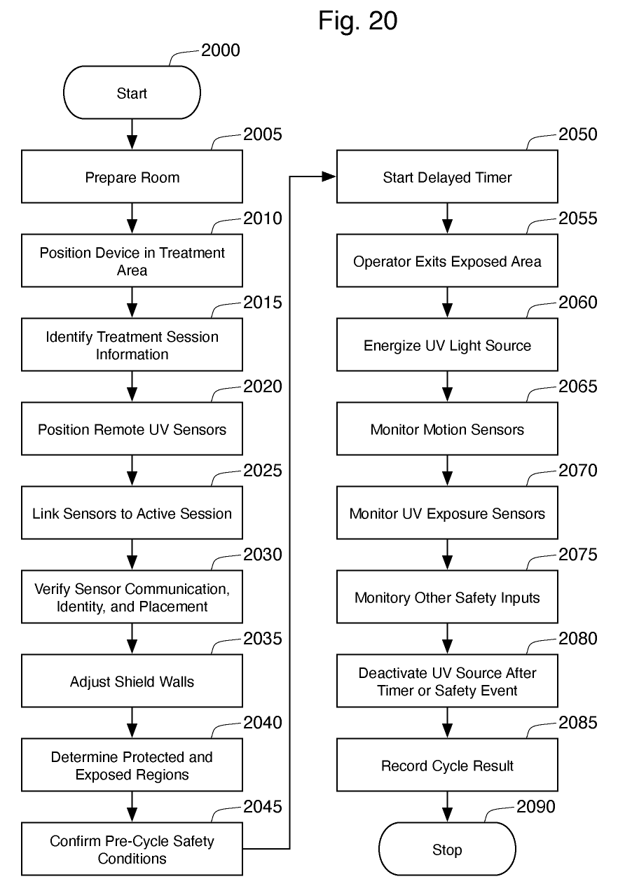

FIG. 20 illustrates a method 2000 for operating a UV sterilization device with one or more remote UV sensors. The method 2000 begins at step 2005, in which the room or other treatment area is prepared for a disinfection cycle. Preparing the room may include wiping down or otherwise cleaning surfaces before ultraviolet treatment is initiated, removing unnecessary items from the treatment area, closing doors or curtains, and covering or removing reflective surfaces such as mirrors, metallic picture frames, polished metal objects, or other surfaces that may reflect ultraviolet light. Reflective surfaces may be covered with a sheet, drape, cloth, disposable cover, UV-attenuating material, or other suitable material. The preparation step may also include providing instructions to the patient 1930 or other persons in the room, such as instructing the patient to remain in a designated location, to avoid moving into an exposed region during operation, and to avoid covering, removing, or repositioning any sensor that will be used during the disinfection cycle. In some embodiments, the graphical user interface may present a preparation checklist or receive confirmation that the room has been prepared before the method proceeds.

At step 2010, the UV sterilization device is positioned in the room 1900 or other treatment area. The device may be placed at a location selected to permit ultraviolet light from the UV light source 1912 to reach surfaces, air, equipment, or other areas intended to be disinfected during the cycle. The device may be rolled, carried, placed, or otherwise moved into the treatment area, depending on the form of the device being used. In some embodiments, the position of the device may be selected by the operator based on the location of the patient 1930, bed 1920, furniture, equipment, doorways, or other features of the room 1900.

At step 2015, treatment session information is identified. The treatment session information may include one or more of a room identifier, patient identifier, device identifier, operator identifier, cycle duration, delayed-start time, intended treatment mode, desired ultraviolet dose, exposure threshold, or other information associated with the disinfection cycle. The treatment session information may be entered through the graphical user interface 1914, selected from stored information, scanned from a barcode or other identifier, received from another system, or otherwise provided to the device 1910. This information may be used to associate the cycle with the correct room, patient, device, and treatment record.

At step 2020, one or more remote UV sensors are positioned in the room 1900 or other treatment area. For example, the personal UV sensor 1940 may be worn by or placed near the patient 1930, the pillow sensor 1950 may be positioned at or near the pillow 1922, and the room sensor 1960 may be positioned at a location in the room intended to receive ultraviolet exposure during the cycle. The number and placement of the sensors may vary depending on the room, the patient location, the areas to be protected, and the areas to be treated. In many instances, the sensors will have been placed earlier, and multiple treatment sessions can take place without re-positioning any of the sensors.

At step 2025, the remote UV sensors are linked to the active treatment session. Linking may include associating one or more sensor identifiers with the device 1910, room 1900, patient 1930, treatment record, or active disinfection cycle. The association may be made through the graphical user interface 1914, a wireless pairing operation, scanning of a sensor identifier, manual entry of an identifier, selection from a displayed list, or another suitable procedure. In one embodiment, the user shall utilize the graphical user interface 1914 to sync the device 1910 to the room 1900 and/or the patient 1930 by manually entering a room or patient identifier. Once linked, the device 1910 may treat the linked sensors as the sensors authorized to provide exposure information for the active session.

At step 2030, the device verifies sensor communication, identity, and placement. Verification of communication may include confirming that the linked sensors are transmitting data to the device 1910. Verification of identity may include confirming that each sensor is the sensor assigned to the active room, patient, device, or treatment session. Verification of placement may include confirming that a sensor has not been covered, removed, turned inward, occluded, or positioned in a manner that would prevent useful exposure monitoring. If communication, identity, or placement cannot be verified, the device may generate an alarm, update the graphical user interface 1914, prevent activation of the UV light source 1912, or require correction before the method proceeds.

In some embodiments, verifying the link between the device 1910 and the remote UV sensors may include confirming that each linked sensor is physically located in the same room or treatment area as the device 1910. For example, before the UV light source 1912 is operated at a disinfection power level, the device 1910 may emit one or more low-power ultraviolet pulses, test bursts, or other coded optical signals. The linked sensors may be required to detect the emitted pulses and transmit confirmation data back to the device 1910. The confirmation data may identify the sensor, indicate the time at which the pulses were detected, indicate the measured intensity or pulse pattern, or otherwise confirm that the sensor received the expected optical signal. If a sensor does not report receipt of the expected pulses, or if an unlinked sensor reports receipt of the pulses, the device 1910 may update the graphical user interface 1914, issue an alarm, prevent activation of the UV light source 1912, or require the operator to repeat the linking procedure.

The pulses may be emitted according to a predetermined or randomly generated pattern so that the device 1910 can distinguish its own verification signal from ultraviolet light emitted by another device operating in a different room. For example, the device 1910 may emit a sequence of short bursts having a selected timing, spacing, duration, intensity, or coded pattern, and each linked sensor may be required to report the same sequence before the sensor is accepted for the active treatment session. This prevents a sensor associated with one room from being mistaken for a sensor associated with another room, particularly when multiple UV sterilization devices are being used at the same time in different rooms. This procedure may also be used to link the sensors to the active session in step 2025, allowing the linking step 2025 and the verification step 2030 to occur simultaneously.

Other link-verification techniques may also be used. For example, the device 1910 and each sensor may exchange a wireless pairing code, session identifier, room identifier, device identifier, patient identifier, rolling code, or encrypted challenge-response message. The device 1910 may verify the received signal strength, time of flight, proximity, network location, or short-range communication range of a sensor to determine whether the sensor is likely located in the same room. The operator may also scan a barcode, QR code, RFID tag, NFC tag, or other identifier on the sensor, patient, room, or device to associate the sensor with the active session. In some embodiments, two or more verification techniques may be used together, such as requiring both wireless pairing and detection of a coded ultraviolet test pulse before the UV light source 1912 is permitted to operate at a disinfection level.

At step 2035, one or more shield walls of the device 1910 are adjusted to define one or more protected regions within the room 1900. The shield walls may be positioned manually by the operator or may be positioned automatically or semi-automatically by the device 1910. The shield walls may be adjusted based on the location of the patient 1930, bed 1920, pillow 1922, personal UV sensor 1940, pillow sensor 1950, room sensor 1960, doorway, caregiver pathway, equipment, or other area to be protected from direct ultraviolet illumination. In some embodiments, the graphical user interface 1914 may present a selected shield position, receive a user selection of a preset shield configuration, or receive user input for manually or incrementally positioning the shield walls.

At step 2040, the device 1910 determines protected and exposed regions within the room 1900. The protected region may correspond to a shadow region in which direct ultraviolet light from the UV light source 1912 is blocked or attenuated by one or more shield walls. The exposed region may correspond to one or more areas that receive direct ultraviolet illumination from the UV light source 1912. The device 1910 may determine the protected and exposed regions based on sensed shield-wall positions, user-entered shield positions, preset shield configurations, UV sensor readings, a calibration routine, or another suitable technique. In some embodiments, the device 1910 may determine whether the personal UV sensor 1940 or pillow sensor 1950 is located in or near a protected region, while the room sensor 1960 is located in a region intended to receive ultraviolet exposure during the disinfection cycle. In some embodiments, the device 1910 does not have physical shield walls, and screens are used to create a protected region.

At step 2045, the device 1910 confirms pre-cycle safety conditions before the UV light source 1912 is energized at a disinfection level. The pre-cycle safety conditions may include confirmation that the room 1900 has been prepared, the device 1910 has been properly positioned, the treatment session information has been entered, the remote UV sensors have been linked to the active session, and communication, identity, and placement of the linked sensors have been verified. The device 1910 may also confirm that the shield walls are connected and positioned as expected, that the protected and exposed regions have been determined, that no unsafe motion or occupancy condition is detected in an exposed region, that any required timer, alarm, GUI, or emergency-stop functions are available, and that no fault condition is present. If one or more pre-cycle safety conditions are not satisfied, the device 1910 may prevent activation of the UV light source 1912, issue an alarm, update the graphical user interface 1914, and require correction before the method proceeds.

At step 2050, the operator initiates the disinfection cycle using the graphical user interface 1914, a remote control, a physical switch, or another user input device. In some embodiments, initiation of the cycle starts a delayed timer that provides a time interval between initiation of the cycle and energization of the UV light source 1912. The delayed timer allows the operator and other persons to leave an exposed area before ultraviolet radiation is emitted at a disinfection level. In other embodiments, a delayed timer is not required because the operator may use a remote control to trigger energization of the UV light source 1912 after the operator has already exited the exposed area or moved to a protected location. When a remote control is used, the remote control may receive and display the same or similar information provided through the graphical user interface 1914, including timer status, cycle status, linked sensor status, shield status, treatment session information, and fault or warning information.

At step 2055, the operator exits the exposed area or moves to a protected location. For example, the operator may leave the room 1900, close a door, move behind a barrier, remain within a protected shadow region, or otherwise move outside the region expected to receive direct ultraviolet illumination. In some embodiments, the operator may remain in the room 1900 during at least part of the disinfection cycle, in which case the operator may wear or carry an operator-associated UV sensor or dosimeter. The operator-associated UV sensor or dosimeter may be linked to the active session in the same manner as the personal UV sensor 1940, pillow sensor 1950, or room sensor 1960, and the device 1910 may monitor exposure data from the operator-associated sensor to determine whether exposure to the operator remains within permitted limits.

At step 2060, the UV light source 1912 is energized if the delayed timer has expired, or if a remote activation command is received after the operator has exited the exposed area or moved to a protected location, and the required safety conditions remain satisfied. The UV light source 1912 may be energized at a selected disinfection power level, wavelength, intensity, or operating mode for treating the room 1900 or a selected portion of the room 1900. Once the UV light source 1912 is energized, the device 1910 may continue to display cycle status through the graphical user interface 1914 and, when a remote control is used, may also provide cycle status, sensor data, alarms, warnings, and fault information to the remote control.

At step 2065, the device 1910 monitors one or more motion sensors during operation of the UV light source 1912. The motion sensors may detect movement of the patient 1930, the operator, a caregiver, or another person within the room 1900. The device 1910 may determine whether detected motion is located within a protected region, near a boundary of a protected region, or within an exposed region. If motion is detected within a protected region, the device 1910 may continue operation of the UV light source 1912. If motion is detected near a boundary or within an exposed region, the device 1910 may issue a warning, update the graphical user interface 1914, reduce output of the UV light source 1912, or deactivate the UV light source 1912.

At step 2070, the device 1910 monitors UV exposure data received from the remote UV sensors associated with the active session, such as the personal UV sensor 1940, pillow sensor 1950, room sensor 1960, or another linked sensor. The device 1910 may compare instantaneous ultraviolet intensity, cumulative ultraviolet dose, sensor status, or threshold information from the sensors with one or more permitted exposure limits or desired treatment levels. For example, the device 1910 may determine whether exposure measured at or near the patient 1930 remains below a permitted limit while also determining whether the room sensor 1960 has received a desired treatment dose. If a linked sensor reports excessive exposure, loss of communication, improper positioning, or another fault condition, the device 1910 may issue an alarm, update the graphical user interface 1914, reduce output of the UV light source 1912, or deactivate the UV light source 1912.

At step 2075, the device 1910 monitors other safety inputs during the disinfection cycle. These safety inputs may include shield-position sensors, shield-connection sensors, proximity sensors, door sensors, tip sensors, temperature sensors, wheel-brake sensors, emergency-stop switches, remote-control inputs, timer status, communication status, or other safety-related signals. Each safety input may be used to determine whether an unsafe condition exists. For example, a proximity sensor may detect a distance between the device 1910, the UV light source 1912, a UV barrier, a patient, an operator, or another object, and an unsafe condition may exist when the detected distance is less than a predetermined minimum distance. Similarly, an unsafe condition may exist when a shield or UV barrier is disconnected, improperly connected, or moved from an expected position. If one of these safety inputs indicates that an unsafe condition exists, the device 1910 may generate an alarm, update the graphical user interface 1914, reduce output of the UV light source 1912, pause the disinfection cycle, or deactivate the UV light source 1912.

At step 2080, the device 1910 deactivates the UV light source 1912 when the disinfection cycle is complete or when a safety event occurs. The disinfection cycle may be complete when a selected timer expires, when a desired cumulative dose is measured by the room sensor 1960, or when another treatment-completion condition is satisfied. A safety event may include unsafe motion, excessive UV exposure at a patient-associated or operator-associated sensor, loss of communication with a linked sensor, improper sensor placement, shield movement or disconnection, proximity detection, a tip condition, an over-temperature condition, activation of an emergency stop, or another fault. The monitoring steps 2065, 2070, and 2075 may occur at the same time or in overlapping time periods while the UV light source 1912 is energized, so that motion, UV exposure, and other safety conditions are evaluated throughout the active disinfection cycle.

At step 2085, the device 1910 records a cycle result for the disinfection cycle. The recorded cycle result may include one or more of a device identifier, room identifier, patient identifier, operator identifier, linked sensor identifiers, cycle start time, cycle end time, selected cycle duration, actual cycle duration, UV light source operating status, cumulative UV exposure measured by one or more linked sensors, cumulative room dose measured by the room sensor 1960, alarms, warnings, fault conditions, shutdown events, emergency-stop events, and whether the cycle was completed or interrupted. The recorded cycle result may be stored in memory of the device 1910, displayed through the graphical user interface 1914, transmitted to a remote control or external system, or exported for recordkeeping, compliance, maintenance, or treatment-verification purposes. In some embodiments, the cycle result may be associated with a patient record, room record, device service record, or facility disinfection log.

In some embodiments, the device 1910 records a room treatment record based on ultraviolet exposure data received from a room sensor 1960 associated with the exposed region. The room sensor 1960 may be purposefully exposed to ultraviolet light during the treatment cycle so that the device 1910 can determine whether a desired treatment dose has been delivered to the exposed region, confirm that the treatment cycle occurred, and store a record of the delivered room treatment. The room treatment record may be associated with room identification information entered through the graphical user interface 1914 before the treatment cycle. The device 1910 may also store a treatment-cycle record associated with the treatment cycle, and the treatment-cycle record may include one or more of a patient identifier, a room identifier, a sensor identifier, a device identifier, a cycle start time, a cycle end time, a cycle duration, a measured ultraviolet dose, cumulative ultraviolet exposure, a treatment-completion indication, an alarm event, a fault event, a shutdown event, or an indication that the treatment cycle was interrupted or completed. The treatment-cycle record, the room treatment record, or both may be displayed, transmitted, stored in memory, or exported.

The data recorded can also include information used to track the usage of device 1910 over time. For example, total activation time for the device 1910 can be tracked in a non-erasable memory in order to track total usage. To the extent replaceable bulbs or other UV sources are used in the device 1910 as the UV light source 1912, separate data can track total usage time for those replaceable components.

At step 2090, the method 2000 ends. After the method 2000 ends, the device 1910 may remain in a safe state with the UV light source 1912 deactivated. The graphical user interface 1914 may display completion status, shutdown reason, recorded exposure information, or instructions for any further action. The device 1910 may then be reset, moved, serviced, or prepared for another disinfection cycle.

The method 2000 may also be applied to a handheld embodiment of ultraviolet treatment device 1910 that is responsive to one or more of the sensors shown in FIG. 19. In such an embodiment, the handheld device 1910 may be placed on a table, cart, counter, floor, stand, or other support surface before the UV light source is activated. One or more UV barriers, such as screening partitions, movable panels, curtains, or other shield structures, may be positioned to create a shadow region for a patient, operator, bed, pillow, or other monitored location while the handheld device is directed toward an exposed treatment area. The UV barriers may or may not be physically attached to the handheld device 1910. After the handheld device 1910 and UV barriers are positioned, the operator may leave the exposed area or move to a protected location before activating the handheld device using a remote control, a delayed timer, or another activation mechanism. During operation, the device 1910 determines whether inappropriate movement has occurred in or near the monitored shadow region, and may also determine whether a sensor positioned in the shadow region has reached a permitted exposure threshold. In response to inappropriate movement or a reached exposure threshold, the device 1910 may issue an alarm, reduce ultraviolet output, or deactivate the device 1910.

The many features and advantages of the invention are apparent from the above description. Numerous modifications and variations will readily occur to those skilled in the art. Since such modifications are possible, the invention is not to be limited to the exact construction and operation illustrated and described. Rather, the present invention should be limited only by the following claims.

What is claimed is:

1. A method of operating an ultraviolet treatment device, the method comprising:
a) positioning an ultraviolet treatment device in a treatment area, the ultraviolet treatment device comprising an ultraviolet light source;
b) positioning a UV barrier to define a shadow region and an exposed region in the treatment area, wherein the UV barrier is configured to block or attenuate ultraviolet light from the ultraviolet light source;
c) associating a remote ultraviolet sensor with the shadow region;
d) initiating a treatment cycle after an operator has exited the exposed region;
e) energizing the ultraviolet light source during the treatment cycle;
f) while the ultraviolet light source is energized, monitoring for motion in the exposed region;
g) while the ultraviolet light source is energized, receiving ultraviolet exposure data from the remote ultraviolet sensor associated with the shadow region; and
h) deactivating the ultraviolet light source in response to detecting motion in the exposed region or determining from the ultraviolet exposure data that ultraviolet exposure in the shadow region has reached a permitted exposure limit.

2. The method of claim 1, wherein the remote ultraviolet sensor comprises a digital dosimeter that measures cumulative ultraviolet exposure, and wherein the ultraviolet exposure data comprises the cumulative ultraviolet exposure measured by the digital dosimeter.

3. The method of claim 1, wherein the remote ultraviolet sensor comprises a patient-worn ultraviolet sensor, and wherein the method further comprises comparing ultraviolet exposure data received from the patient-worn ultraviolet sensor to a preset safety value.

4. The method of claim 1, wherein associating the remote ultraviolet sensor with the shadow region comprises wirelessly linking the remote ultraviolet sensor to the ultraviolet treatment device, and wherein the ultraviolet exposure data is wirelessly received.

5. The method of claim 1, further comprising receiving ultraviolet exposure data from a room sensor associated with the exposed region, comparing the ultraviolet exposure data received from the room sensor to a desired treatment dose, and determining whether the desired treatment dose has been delivered to the exposed region.

6. The method of claim 5, further comprising recording a room treatment record indicating that the desired treatment dose was delivered.

7. The method of claim 1, further comprising updating a patient cumulative exposure record with the ultraviolet exposure data received during the treatment cycle, the patient cumulative exposure record including ultraviolet exposure accumulated during a plurality of treatment cycles for a patient, wherein determining from the ultraviolet exposure data that ultraviolet exposure in the shadow region has reached the permitted exposure limit comprises determining that the patient cumulative exposure record, as updated with the ultraviolet exposure data received during the treatment cycle, has reached the permitted exposure limit.

8. The method of claim 1, further comprising storing a treatment-cycle record associated with the treatment cycle, wherein the treatment-cycle record includes a patient identifier, a room identifier, a sensor identifier, and a device identifier.

9. The method of claim 1, further comprising performing a calibration function before energizing the ultraviolet light source, wherein the calibration function confirms operation of the remote ultraviolet sensor and confirms that the ultraviolet treatment device is in a safe condition for the treatment cycle.

10. The method of claim 1, further comprising, while the ultraviolet light source is energized, providing feedback through a graphical user interface regarding ultraviolet exposure data received from the remote ultraviolet sensor.

11. The method of claim 1, further comprising, while the ultraviolet light source is energized, monitoring a plurality of safety sensors to determine whether an unsafe condition exists, the plurality of safety sensors including a proximity sensor and a shield-connection sensor, and deactivating the ultraviolet light source in response to a signal from one of the plurality of safety sensors indicating that the unsafe condition exists.

12. The method of claim 1, further comprising deactivating the ultraviolet light source in response to actuation of a manually operable shutdown switch.

13. The method of claim 1, wherein the UV barrier comprises a plurality of shield walls movably attached to the ultraviolet treatment device.

14. The method of claim 1, wherein the ultraviolet treatment device is a portable ultraviolet treatment device positioned on a support surface during the treatment cycle.

15. An ultraviolet treatment system comprising:
a) an ultraviolet treatment device comprising an ultraviolet light source;
b) a UV barrier configured to define a shadow region and an exposed region in a treatment area, wherein the UV barrier is configured to block or attenuate ultraviolet light from the ultraviolet light source;
c) a remote ultraviolet sensor associated with the shadow region and configured to generate ultraviolet exposure data for the shadow region; and
d) a controller configured to
i) energize the ultraviolet light source during a treatment cycle,
ii) receive the ultraviolet exposure data from the remote ultraviolet sensor while the ultraviolet light source is energized,
iii) monitor for motion in the exposed region while the ultraviolet light source is energized, and
iv) deactivate the ultraviolet light source in response to detecting motion in the exposed region or in response to determining from the ultraviolet exposure data that ultraviolet exposure in the shadow region has reached a permitted exposure limit.

16. The ultraviolet treatment system of claim 15, wherein the remote ultraviolet sensor comprises a patient-worn digital dosimeter configured to measure cumulative ultraviolet exposure.

17. The ultraviolet treatment system of claim 15, wherein the controller is configured to update a patient cumulative exposure record with the ultraviolet exposure data received during the treatment cycle, the patient cumulative exposure record including ultraviolet exposure accumulated during a plurality of treatment cycles for a patient, and wherein the controller is configured to deactivate the ultraviolet light source when the patient cumulative exposure record, as updated with the ultraviolet exposure data received during the treatment cycle, has reached the permitted exposure limit.

18. The ultraviolet treatment system of claim 15, further comprising a room sensor associated with the exposed region and configured to generate room ultraviolet exposure data, wherein the controller is configured to determine from the room ultraviolet exposure data that a desired treatment dose has been delivered to the exposed region.

19. The ultraviolet treatment system of claim 15, further comprising a graphical user interface, wherein the controller is configured to store a treatment-cycle record associated with the treatment cycle, the treatment-cycle record including a patient identifier, a room identifier, a sensor identifier, and a device identifier, and wherein the graphical user interface is configured to provide feedback regarding the ultraviolet exposure data received from the remote ultraviolet sensor.

20. The ultraviolet treatment system of claim 15, wherein the UV barrier comprises a plurality of shield walls movably attached to the ultraviolet treatment device, further comprising safety sensors including a proximity sensor and a shield-connection sensor, further wherein the controller is configured to deactivate the ultraviolet light source in response to a signal from the safety sensors.
